(19) United States
(12) Patent Application Publication
(10) Pub. No.: US 2022/0301708 A1
(43) Pub. Date: Sep. 22, 2022

(54) DIABETES RISK EARLY WARNING SYSTEM

(71) Applicant: LINGNAN NORMAL UNIVERSITY, Zhanjiang, Guangdong (CN)

(72) Inventors: Xiue GAO, Zhanjiang, Guangdong (CN); Bo CHEN, Zhanjiang, Guangdong (CN); Shifeng CHEN, Zhanjiang, Guangdong (CN); Haitao SANG, Zhanjiang, Guangdong (CN)

(21) Appl. No.: 16/967,620

(22) PCT Filed: Mar. 19, 2020
(86) PCT No.: PCT/CN2020/080251

§ 371 Date: Aug. 5, 2020

(30) Foreign Application Priority Data
Apr. 18, 2019 (CN) 201910314236.5
Apr. 25, 2019 (CN) 20190340600.5

(51) Int. Cl. G16H 50/20; G16H 50/70

(52) U.S. Cl. CPC G16H 50/20; G16H 50/70

(57) ABSTRACT

The present invention relates to a diabetes early warning system. The system comprises: a memory; and a first processor, which is based on improved k-means clustering, coupled to the memory, and configured to: according to selected first clustering centroids, obtain stable centroids for individual clusters, and put them in a diabetes piecewise function, thereby obtaining a diabetes early warning model, wherein the first clustering centroid is selected by selecting a data set, defining a clustering cluster number k and a neighborhood radius ε, and selecting a sample point on which a sum of distances between a sample point Xᵢ and a sample is the greatest as the first clustering centroid, so as to make the first clustering centroid fall in a central portion of the corresponding cluster. The present invention improves the clustering centroid method, establishes a diabetes piecewise function early warning model, improves the diabetes early warning ability, and provides a basis for the diagnosis and treatment of diabetes at different stages. Starting from the characteristics of the diabetes data set, the key feature variables of diabetes are selected to simplify the diabetes prediction model; and the accuracy of the diabetes prediction model is improved, thereby helping to provide accurate diabetes prevention and treatment measures.

### FIELD

The present invention relates to medical informatization, and more particularly to a diabetes early warning system.

### DESCRIPTION OF RELATED ART

Extensive researches on various aspects of diabetes (e.g. diagnosis, pathophysiology, treatment processes, etc.) conducted by researchers have brought about a huge amount of related data. For example, China Patent Application No. CN107403072A published on Nov. 28, 2017 discloses a diabetes prediction and warning method based on machine learning. The known method uses K-means algorithms and logistic regression algorithms to build a bilayer forecast analysis model that conducts clustering and classification successively. The K-means algorithms are capable of clustering analysis unlabeled data sets. For selection of the initial clustering centroid, the known method seeks for a stable initial clustering centroid by introducing a layered algorithm, namely a next-level logistic regression algorithm. This, however, leads to significantly increased calculation loads for the algorithm. Besides, setting threshold based on empirical solution-seeking breaks convergence of the algorithm, eventually causing difficulty in getting stable clustering results.

On the other hand, the increase of data features in diabetes prediction models and data dimensionality increases non-critical information and redundant information, making prediction models more and more complicated. This hinders conventional prediction methods from being used in diabetes prediction directly. Some existing papers have proposed some solutions for this problem. For example, “The Application of Lasso and Its Related Methods in Multiple Linear Regression Model” written by K E Zheng-lin et al. of Beijing Jiaotong University is about application of Lasso method and its related method to selection of variables for multiple linear regression models. According to the paper, conventional LARS algorithms are used for selection of variables used in multiple linear regression models, and selection of variables is realized using diabetes statistical data and analogically generated multivariate statistical data. However, when used to figure out a Lasso regression coefficient, these conventional LARS algorithms are disadvantageous for slow approaching and poor accuracy. In addition, since the iteration direction in LARS algorithms depends on the residual of the target, the algorithms are highly sensitive to noises in samples. These make it difficult to use LARS algorithms directly to diabetes prediction applications with increased data features and data dimensions.

### SUMMARY OF THE INVENTION

In view of the problem about inconsistent clustering results of randomly selected initial clustering centroids in conventional k-means clustering algorithms, the objective of the present invention is to provide a diabetes early warning system based on improved k-means algorithms that are optimized in terms of initial clustering centroid. The present invention also provides a method that incorporates diabetes piecewise functions to improve k-means clustering diabetes early warning models. In addition, the present invention according to PCA principle component analysis, in consideration of the effects of different diabetes features on prediction results, provides a method for improving computation of the relationship between feature independent variables and dependent variables, thereby simplifying the diabetes prediction model and enabling feature-weight-based LARS prediction of diabetes.

The diabetes early warning system of the present invention includes at least one processor, a system memory and at least one computer-readable storage medium. The at least one computer-readable storage medium is loaded with computer-executable instructions that are used to make the processor realize all aspects of the present invention. The at least one processor is used to execute the computer-executable instructions. The flowcharts and block diagrams in the accompanying drawings illustrate structures, functions and operations of systems, methods and computer program products according to embodiments of the present invention. Therein, every block in a flowchart or block diagram may represent a part of a module, a program segment or an instruction. A part of a module, program segment or instruction includes one or more executable instructions used to realize specified logical functions. Every block in a block diagram and/or a flowchart, and combinations of blocks of a block diagram and/or a flowchart may be implemented through a hardware-based special system that executes specified functions or motions, or may be implemented through a combination of special hardware and computer instructions. The flowcharts and/or block diagrams with reference to methods, devices (systems) and computer program products according to the embodiments of the present invention depict various aspects of the present invention. It is to be understood that every block in the flowcharts and/or block diagrams and any combination of the blocks in the flowcharts and/or block diagrams may be implemented using computer-readable program instructions. The aforementioned computer-readable storage medium may be tangible equipment that holds and stores instructions to be used by instruction executing equipment. Examples of the computer-readable storage medium include but are not limited to an electrical memory, a magnetic memory, an optical memory, an electromagnetic memory, a semiconductor memory or any combination thereof. The processor is a functional unit that explains and executes instructions, and is also known as a central processor or a CPU. It acts as an operational and control core of a computer system, and is the initial execution unit for formation processing and program operation. The memory may be a read-only memory (ROM), a random-access memory (RAM), an external memory such as a hard disk, a floppy disk, a compact disk, USB flash disk, or a storage server.

To achieve the foregoing objective, the present invention adopts the following technical schemes:

A diabetes early warning system, comprising: a memory; a first processor, coupled to the memory and configured to: according to selected first clustering centroids, obtain stable centroids for individual clusters, and put them in a diabetes piecewise function, thereby obtaining a diabetes early warning model, wherein the first clustering centroid is selected by selecting a data set, defining a clustering cluster number k and a radius ε, and selecting a point on which a sum of distances between a sample point Xᵢ and samples, so as to make the first clustering centroid fall in a central portion of the corresponding cluster;

A device, used to obtain a regression coefficient for a diabetes prediction model by means of a LARS algorithm based on feature weights, and comprising modules that are configured to execute at least one step of the feature-weight based LARS diabetes prediction method, respectively;

A diabetes early warning system, the system include: a memory; a second processor, coupled to the memory and configured to: calculate an independent variable feature weight vector and an original relationship vector; based on the independent variable feature weight vector and the original relationship vector, output a regression coefficient ω of a LARS diabetes model based on the feature weight;

A device, used to build a diabetes prediction model, the device comprising modules configured to execute at least one step of the method for building the diabetes prediction model; and

A diabetes early warning system, comprising: a memory; at least one processor, coupled to the memory and configured to: according to selected first clustering centroids, obtain stable centroids for individual clusters, and put them in a diabetes piecewise function, so as to obtain a diabetes prediction model, wherein the first clustering centroid is selected by selecting a data set, defining a clustering cluster number k and a radius ε, and selecting a point on which a sum of distances between a sample point Xi and samples, so as to make the first clustering centroid fall in a central portion of the corresponding cluster; the system calculating an independent variable feature weight vector and an original relationship vector; and based on the independent variable feature weight vector and the original relationship vector outputting a regression coefficient ω of the diabetes prediction model.

### BRIEF DESCRIPTION OF THE DRAWINGS

FIG. 1 is a flowchart of a feature-weight-based LARS diabetes prediction method according to one embodiment of the present invention;

FIG. 2 is a solution graph according to the standard LARS algorithm;

FIG. 3 is a solution graph according to a LARS algorithm incorporating feature weights as disclosed in one embodiment of the present invention;

FIG. 4 shows variations of the regression variable ω according to the standard LARS algorithm;

FIG. 5 shows variations of the regression variable ω according to the disclosed feature-weight-based LARS algorithm;

FIG. 6 shows ACC variations vs. iteration times according to the standard LARS algorithm and the disclosed feature-weight-based LARS algorithm;

FIG. 7 shows ROC variations vs. iteration times according to the standard LARS algorithm and the disclosed feature-weight-based LARS algorithm;

FIG. 8 is a flowchart of an algorithm of a method for improving a k-means clustering diabetes early warning model according to one embodiment of the present invention;

FIG. 9 compares average rates of convergence of different algorithms on a new diabetes data set according to one embodiment of the present invention; and

FIG. 10 is a line chart that compares average ARIs of multiple clustering results of different algorithms on a new diabetes data set according to one embodiment of the present invention; and

FIG. 11 illustrates simplified module connection of a preferred diabetes early warning system according to the present invention.

### DETAILED DESCRIPTION OF THE INVENTION

The invention as well as a preferred mode of use, further objectives and advantages thereof will be best understood by reference to the following detailed description of illustrative embodiments when read in conjunction with the accompanying drawings. The following embodiments are used to describe the present invention but are not required to limit the scope of the present invention.

### Embodiment 1

According to machine learning and PCA (Principal Component Analysis) theories, a multidimensional sample usually has a few key features or principle components. Among the numerous features of diabetes, only a few are key features. Some studies found that prediction models with better generalization ability as represented by key features can be obtained by the use of LARS algorithms. Generalization ability refers to a quality with which a trained network can produce suitable output even if the input is not a sample set. The machine learning algorithm is realized using a linear regression algorithm. However, over-fitting is an unavoidable issue in use of a linear regression algorithm. The more a model is trained, the more the model matches the training data, and gradually loses its ability to predict when processing new data. Problems of using conventional LARS algorithms to figure Lasso regression coefficients include slow approaching and poor accuracy. In addition, since the iteration direction in LARS algorithms depends on the residual of the target, the algorithms are highly sensitive to noises in samples.

To address the foregoing problems, the present invention incorporates feature weights obtained using a PCA algorithm in the LARS algorithm solving process. Due to different weights of features, the possibility that a feature independent variable is selected has changed, and this may speed up the process the algorithm approaches key features, thereby improving the algorithm solving speed and accuracy. Besides, since the PCA algorithm poses restriction on variations of the independent variables, the model has better robustness. The system of the present invention is used to build a diabetes prediction model. The system at least comprises a memory and a second processor 2. The second processor 2 is coupled to the memory and is configured to execute at least one step of the feature-weight-based LARS diabetes prediction method. Therein, the feature-weight-based LARS diabetes prediction method at least comprises at least one of the following steps.

The first thing to do is to define a diabetes data set feature matrix:

$$X = {( {x}_{1} , {x}_{2} , ⋯ , {x}_{m} )}^{T} = [ \begin{matrix}{x}_{11} & {x}_{12} & ⋯ & {x}_{1 n} \\ {x}_{21} & {x}_{22} & ⋯ & {x}_{2 n} \\ ⋮ & ⋮ & ⋱ & ⋮ \\ {x}_{m 1} & {x}_{m 2} & ⋯ & {x}_{mn}\end{matrix} ] ∈ {R}^{m \times n} .$$

This is a matrix composed of m n-dimensional features, where xₖ₁,xₖ₂, . . . ,xₖₙ are independent variables of individual features.

Tags of actual results: y=(y₁,y₂, . . . ,yₘ)^{T}.

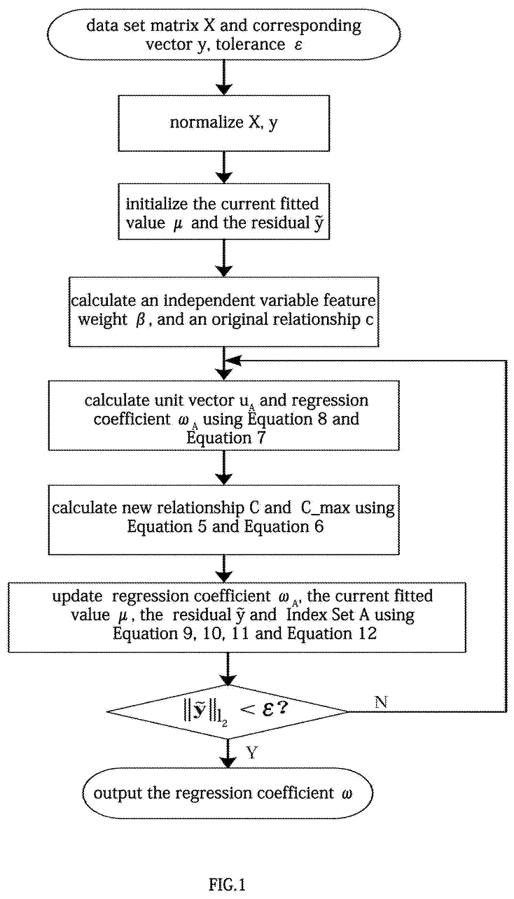

Referring to FIG. 1, the present invention provides a feature-weight-based LARS diabetes prediction method, which comprises the following steps:

Step 1 involves normalizing a diabetes data set matrix X, so that the range values of different features of the diabetes data set are mapped into the same, fixed range of 0-1. The current fitted value and the current residual are initialized. The fitted value refers to the fitting to the actual result in every iteration, initialized to 0. The residual is the difference between the actual result and the fitted value, calculated using Equation 1:

\tilde{y}=y−μ (1)

where y is the actual result label vector, and μ is the current fitted value.

Step 2 involves calculating every feature independent variable initial weight using Equation 3:

$${β}_{i} = \frac{{φ}_{i}}{{\sum }_{k = 1}^{n} {φ}_{k}} \qquad ( 3 )$$

whereφᵢ is the feature value of the feature equation |φI−R|=0. In the feature equation, R is a covariance matrix of the diabetes data set matrix X , calculated using Equation 4:

$$R = [ \begin{matrix}{r}_{11} & {r}_{12} & ⋯ & {r}_{1 n} \\ {r}_{21} & {r}_{22} & ⋯ & {r}_{2 n} \\ ⋮ & ⋮ & ⋱ & ⋮ \\ {r}_{m 1} & {r}_{m 2} & ⋯ & {r}_{mn}\end{matrix} ] \qquad ( 4 )$$

where

$${r}_{ij} = {r}_{ji} = \frac{{\sum }_{k = 1}^{m} ( {x}_{ki} - {θ}_{i} ) ( {x}_{kj} - {θ}_{j} )}{\sqrt{{\sum }_{k = 1}^{m} {( {x}_{ki} - {θ}_{i} )}^{2} {\sum }_{k = 1}^{m} {( {x}_{kj} - {θ}_{j} )}^{2}}} ,$$

and θᵢ is a mean of the ith feature.

Then the initial relationship between every feature independent variable and the actual result is calculated using Equation 2:

c=X^{T}y (2)

The Lasso model may produce a sparse solution. The obtained diabetes prediction model has better generalization ability. The LARS algorithm can be used to solve the Lasso model, but when applied to a diabetes data set, it is disadvantageous because of slow approaching and poor accuracy. The present invention improves the LARS algorithm by means of PCA, so as to obtain weights of different features and determine how important each feature independent variable is, thereby providing a feature-weight-based LARS diabetes prediction method.

Referring to FIG. 3, Step 3 involves extracting a row vector having the same direction as y from X, and making it X_{A}. X_{A} is the row vector in Index Set A extracted from X.

Afterward, the angle bisector line U_{A} of the vector in X_{A} is calculated using Equation 8 and Equation 7:

G_{A}=X^{T}_{A}X_{A}, A_{A}=(1^{T}_{A}G⁻¹_{A}1_{A})^{-1/2} (7)

ω_{A}=A_{A}G⁻¹_{A}1_{A}, u_{A}=X_{A}ω_{A} (8)

where, 1_{A} is the row vector whose all elements in the k dimension are 1, and k is the number of elements in A.

The new relationship is calculated using Equation 5:

C=c^{T}β (5)

where c=X^{T}(y−μ_{A}), μ_{A} is the fitted value of the previous step, β is the measure vector of each weight obtained using the PCA algorithm, so the maximum of C can be obtained using Equation 6:

C_max=max{|C|} (6)

In Step 4, the regression coefficient, the current fitted value and the current residual can be updated. The regression coefficient vector is updated using Equation 9:

ω_{A}=ω_{A}+γω_{A} (9)

The fitted value vector is calculated using Equation 10:

μ_{A}=μ_{A}+γu_{A} (10)

The residual vector is calculated using Equation 11:

\tilde{y}(γ)=y−μ_{A}−γu_{A} (11)

where γ is the step length along the angle bisector line u_{A}, and assuming that a=X^{T}u_{A}, γcan be calculated using Equation 12:

$$γ = {min}_{i ∉ A}^{+} \{ \frac{C\_max - {C}_{i}}{{A}_{A} - {a}_{i}} , \frac{C\_max + {C}_{i}}{{A}_{A} + {a}_{i}} \} \qquad ( 12 )$$

where the plus sign over min means only the minimum of positive numbers in the set is calculated, Cᵢ and aᵢ are the i^{th} element in C and a respectively, and i is such selected that \tilde{y}(γ)=y−μ_{A}−γu_{A} can get the i with the minimum value.

In Step 5, it is to be determined that whether the L2 norm of the residual in Step 4 is smaller than a certain tolerance, and the process ends by outputting the regression coefficient if yes, or Step 3 through Step 5 are repeated if no.

Therein, the regression coefficient in the regression equation is a parameter representing how much the independent variable x influences the dependent variable y. The larger the regression coefficient is, the greater the influence of x on y is. A positive regression coefficient means that y increases with x, and a negative regression coefficient means that y decreases as x increases. For example, in the regression equation Y=bX+a, the slope b is regarded as the regression coefficient, meaning that when X changes for one unit, Y changes for b units in average.

By using the Lasso model, feature selection can be performed to get diabetes key features. Then diabetes key feature variables are screened out according to PCA. This simplifies the diabetes prediction model and improves the diabetes prediction model in terms of accuracy, and prepares for more accurate diabetes prevention and treatment measures.

The disclosed diabetes prediction method is compared with the standard LARS method using regression coefficient paths, prediction accuracy (ACC) curves and receiver operating characteristic (ROC) curves of their models as criteria. Therein, the regression coefficient paths show how the feature independent variable coefficients vary in an intuitive way, and the ACC curves facilitate intuitive comparison of algorithms in terms of approaching speed and accuracy. ROC curves server as a tool for measurement of unequilibrium issues, for which the larger area under the curve means that the model is better.

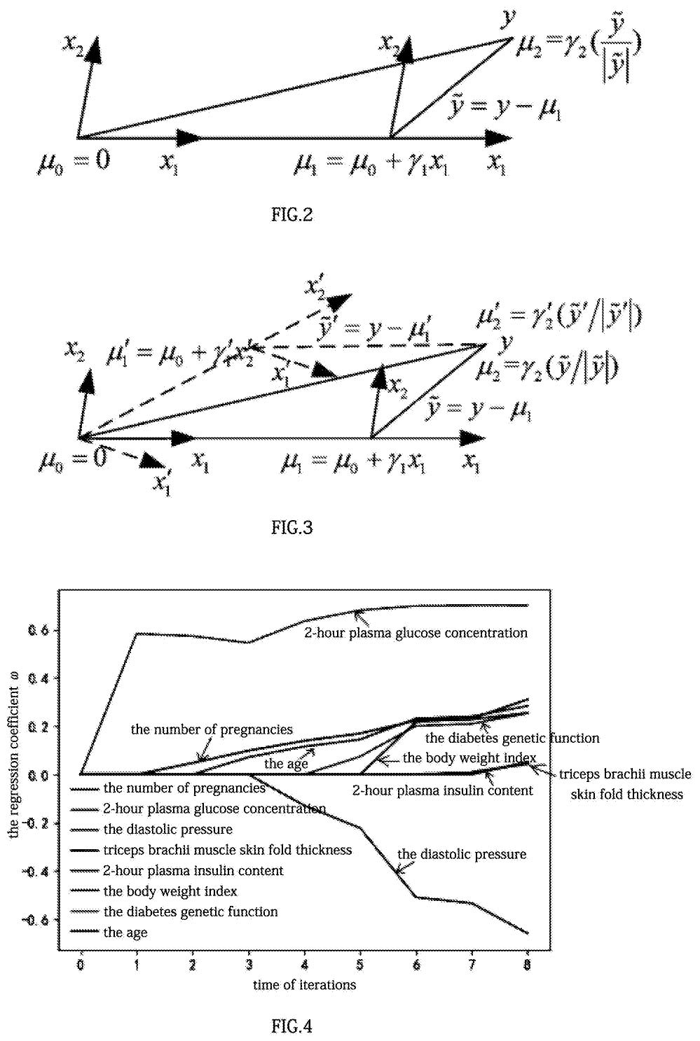

Intuitionally, the standard LARS algorithm works by finding out the independent variable xₖ most correlative to y, using it to approach y, until another x₁ that has a relationship with y equal to the relationship between xₖ and y appears, and then starting to approach y in the direction of the angular bisector between xₖ and x₁. Similarly, when a third xₚ having a large enough relationship with the dependent variable appears, it is added to the approach queue. The common direction of the angular bisectors of the three vectors (the phrase “angular bisector” refers to a halving line of a high vector) is taken and so on, until the residual is small enough or until all the independent variables have been obtained, at which point the algorithm ends. As shown in FIG. 2, initially, the relationship between x₁ and y was high, so x₁ was used for approach, until y appeared on the angular bisector between x₁ and x₂. Then the direction of the angular bisector between x₁ and x₂ was used to approach the dependent variable y. The standard LARS algorithm retains complexity of the forward selection algorithm, using up to m steps, wherein m is the number of the independent variables, while ensuring the optimal results in the independent variable subspace. FIG. 2 and FIG. 3 are solution graphs related to the standard LARS algorithm and the LARS algorithm incorporating feature weights, respectively. Taking two feature independent variables for example, relationship calculation was performed before every time of approach. As shown in FIG. 3, after the feature weights were added, the possibility for the two feature independent variables to be selected was changed due to different weights. Also changed was the approaching direction, and this eventually led to the varied regression coefficients.

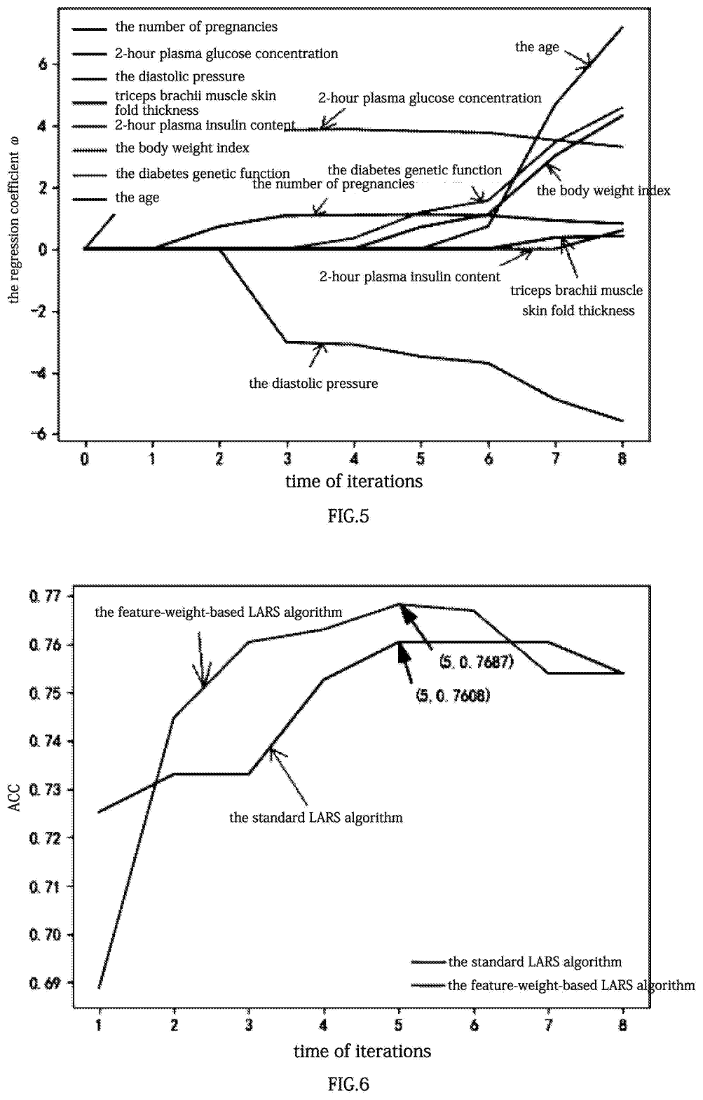

FIG. 4 and FIG. 5 show variations of the regression variable ω related to the standard LARS algorithm and the feature-weight-based LARS algorithm, respectively. The ordinate indicates the size of ω, and the abscissa shows the time of iterations. From comparison between FIG. 4 and FIG. 5, it is clear that all the 8 feature independent variables of the feature-weight-based LARS algorithm had changes in their regression coefficient paths. Since the initial weights were obtained using the PCA algorithm, variations of the individual independent variables were restricted, so the difference in the regression coefficients was not significant, meaning that the model became more robust. Second, since an independent variable with a larger weight has its regression coefficient increasing faster, the results were more reasonable, for example, the diabetes genetic function and the age have bigger influence to the possibility of getting the disease than the number of pregnancies. Therein, 2-hour plasma glucose concentration is for checking the functionality of β cells and regulation of blood sugar of the organism, and is extensively used in clinical practice. The normal range of the diastolic pressure of an adult is <90 mmHg(12 kpa). The triceps brachii muscle skin-fold thickness is used to determine obesity. A man having his triceps brachii muscle skin fold thickness greater than 10.4 mm is regarded as obese, and the number for a female is 17.5 mm. 2-hour plasma insulin, plasma insulin is the only hormone in an organism that decreases blood sugar, and is the only hormone that facilitates synthesis of all three of glycogen, fat and proteins. The normal range for an adult is 29˜172 pmol/L, and for a person order than 60 the range is 42˜243 pmol/L. As to the body weight index, the normal range for an adult is 21-23 kg/m², wherein a person with the BMI falling in the range of 18.5-24.9 kg/m² is regarded as healthy, and a person with the BMI falling in the range of 25.0-29.9 kg/m² is regarded as highly susceptible to diabetes. BMI=body weight in kg/(body height in m)².

FIG. 6 provides ACC curves varying with iterations according to two algorithms. As shown, the feature-weight-based LARS was superior to the standard LARS in terms of approaching speed and accuracy. Before reaching the optimal ACC, the feature-weight-based LARS algorithm performed three iterations while the standard LARS algorithm performed five iterations. Both the feature-weight-based LARS and the standard LARS had their ACCs reaching the zenith, with the ACC of the feature-weight-based LARS higher than that of the standard LARS for about 0.8 percent. The drawing also shows that the two curves both had their ACCs descended during the last few iterations. Since the level of compression of the regression coefficient decreased as the time of iterations increased, when the coefficient of compressibility a became 0, the regression coefficient could not be compressed anymore, so the ACC decreased instead. Table 1 and Table 2 the values of the coefficient of compressibility α and the values of the regression coefficient ω corresponding to different times of iterations n according to the standard LARS and the feature-weight-based LARS, respectively. As reflected in the two tables, the both have three 0s of ω in the fifth iteration, indicating that the regression coefficients of the three independent variables in the final model were 0. At this time, the model was well simplified and the highest ACC was reached.

TABLE 1
coefficient of compressibility α and regression coefficient corresponding
to different times of iterations n according to the standard LARS

| n | α | ω | | | | | | | |
|---|---|---|---|---|---|---|---|---|---|
| 1 | 0.01729 | 0.0 | 0.5835 | 0.0 | 0.0 | 0.0 | 0.0 | 0.0 | 0.0 |
| 2 | 0.01436 | 0.0474 | 0.5739 | 0.0 | 0.0 | 0.0 | 0.0 | 0.0 | 0.0 |
| 3 | 0.00902 | 0.0981 | 0.5454 | 0.0 | 0.0 | 0.0 | 0.0 | 0.0 | 0.0714 |
| 4 | 0.00665 | 0.1395 | 0.6352 | −0.1287 | 0.0 | 0.0 | 0.0 | 0.0 | 0.1156 |
| 5 | 0.00507 | 0.1699 | 0.6807 | −0.2218 | 0.0 | 0.0 | 0.0 | 0.0741 | 0.1450 |
| 6 | 0.00171 | 0.2235 | 0.6983 | −0.5094 | 0.0 | 0.0 | 0.2182 | 0.2011 | 0.2317 |
| 7 | 0.00144 | 0.2281 | 0.7004 | −0.5340 | 0.0100 | 0.0 | 0.2319 | 0.2099 | 0.2399 |
| 8 | 0.0 | 0.2549 | 0.7001 | −0.6588 | 0.0433 | 0.0524 | 0.3109 | 0.2536 | 0.2838 |

TABLE 2
coefficient of compressibility α and regression coefficient corresponding
to different times of iterations n according to the feature-weight-based LARS

| n | α | ω | | | | | | | |
|---|---|---|---|---|---|---|---|---|---|
| 1 | 0.00733 | 0.0 | 2.5476 | 0.0 | 0.0 | 0.0 | 0.0 | 0.0 | 0.0 |
| 2 | 0.00139 | 0.7269 | 2.4992 | 0.0 | 0.0 | 0.0 | 0.0 | 0.0 | 0.0 |
| 3 | 0.00034 | 1.0803 | 3.8571 | −3.0213 | 0.0 | 0.0 | 0.0 | 0.0 | 0.0 |
| 4 | 0.00032 | 1.0895 | 3.8667 | −3.0972 | 0.0 | 0.0 | 0.0 | 0.3441 | 0.0 |
| 5 | 0.00026 | 1.1160 | 3.8094 | −3.4854 | 0.0 | 0.0 | 0.7060 | 1.1857 | 0.0 |
| 6 | 0.00024 | 1.0834 | 3.7602 | −3.7009 | 0.0 | 0.0 | 1.1092 | 1.5628 | 0.7283 |
| 7 | 0.00009 | 0.9148 | 3.5126 | −4.8735 | 0.3713 | 0.0 | 3.0203 | 3.4501 | 4.6804 |
| 8 | 0.0 | 0.8174 | 3.3042 | −5.5739 | 0.4185 | 0.5987 | 4.3109 | 4.5767 | 7.1743 |

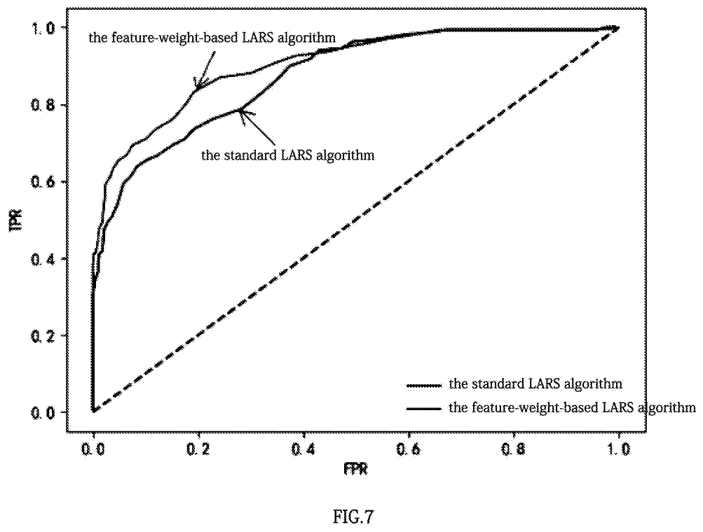

FIG. 7 shows 100 false positive rates and true positive rates calculated when the threshold t increased from 0 to 1 with a step length of 0.01. Between the resulting ROC curves of the two LARS algorithms, the red dotted line represents an ROC curve randomly guessed. It is clear that the ROC curve related to the feature-weight-based LARS is closer to the upper left corner. The areas under the ROC curves AUC were calculated, and the AUC related to the feature-weight-based LARS is 0.8953, while the AUC related to the standard LARS is 0.8664. The feature-weight-based LARS algorithm produced the highest AUC.

To sum up, the diabetes prediction model obtained using the feature-weight-based LARS algorithm has a higher ACC than that of the standard LARS, and approached to the optimal model faster as compared to the standard LARS. Additionally, for the diabetes unequilibrium samples processed herein, the AUC value of the ROC curve of the feature-weight-based LARS algorithm is higher than that of the standard LARS. Thus, the feature-weight-based LARS algorithm is superior to the standard LARS algorithm when solving the diabetes prediction model.

### Embodiment 2

The present embodiment provides further improvements to the feature-weight-based second processor 2 of Embodiment 1, and what is identical to its counterpart in the previous embodiment will not be repeated in the following description. Specifically, the present embodiment provides a diabetes early warning system, the system at least comprises a second processor 2 as described in Embodiment 1, a memory coupled to the processor, and an interface 5 therebetween.

The diabetes early warning system is applicable to rehabilitation exercise risk management for patients highly susceptible to diabetes.

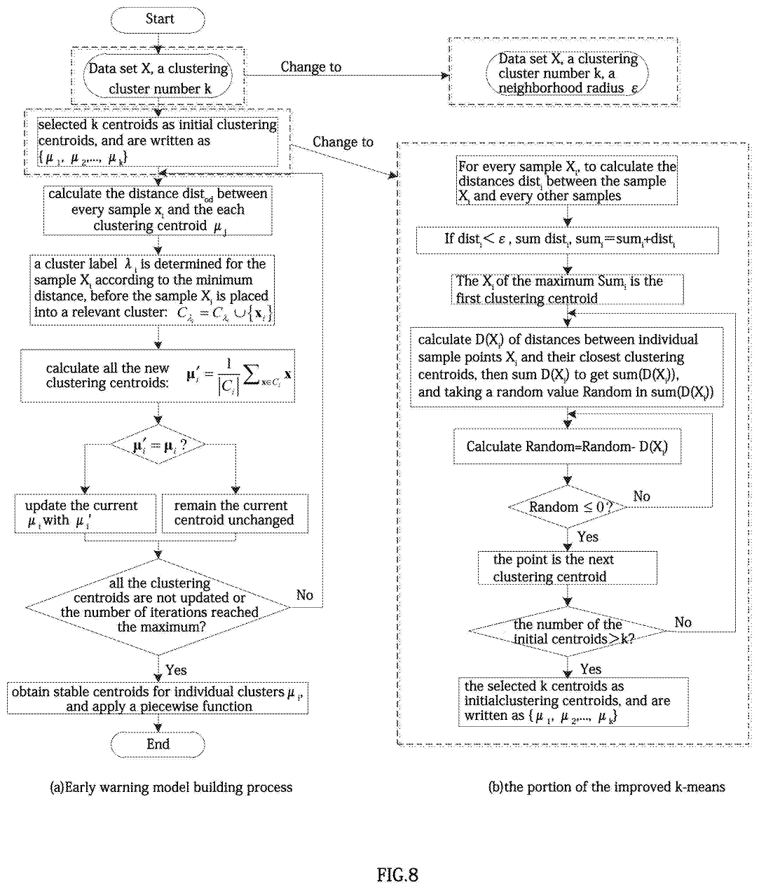

Referring to FIG. 8, the diabetes early warning system at least comprises a sensor module, a second processor 2 and an exercise-regimen-adjusting module. The sensor module is configured to collect initial data of the diabetes early warning system, system parameters and user data related to the user. The second processor 2 is configured to identify user exercise diabetes risk based on the salient features that are screened out from the data set collected by the sensor module according to machine learning and also based on the relationship between the extracted exercise monitoring data and autonomous behavior. The exercise-regimen-adjusting module is configured to dynamically adjust configuration parameters of an exercise regimen by identifying exercise diabetes risk based on the relationship between the exercise monitoring data determined by the second processor 2 through analysis and the autonomous behavior.

The sensor module is now described in detail. The sensor module is used to collect the initial data of the diabetes early warning system, the parameters used by the system and user data related to the user. The collected data are stored in a database, and the integrated data set is transmitted to the second processor 2. The sensor module is built mainly through the following two forms: such as a smart watch to be worn by a user at his/her wrist, used to collect or monitor the physiological monitoring parameters of the user throughout the period of exercise and exercise monitoring data. Preferably, the smart electronic device 4 may be a small portable self-mixing coherent laser radar invasive blood sugar measuring system as disclosed in China Patent Application Publication No. CN202051710U published on Nov. 30, 2011. The known system combines laser radar frequency modulated continuous wave technology and self-mixing coherent technology to realize invasive blood sugar measurement for users. The exercise monitoring data include sedentariness duration, exercise intensity, exercise time, exercise duration, exercise frequency, exercise type and so on. The mechanical equipment is equipped with a pinch sensor, a grip sensor, a torque sensor, a myoelectricity acquisition device, at least one digital transmitter, and a joint movement degree sensor, for collecting exercise ability data about the upper limbs or lower limbs of a user. The analog voltage signals collected by the pinch sensor/grip sensor/torque sensor are processed and converted by a digital transmitter into digital voltage signals. The myoelectricity acquisition device includes electrodes, a low-pass filter circuit module, a bandpass-amplifier circuit module and an analog-to-digital converter circuit module. The biological electromyography signals at human body surface are collected by the myoelectricity acquisition device and converted into digital voltage signals. The joint movement degree sensor may be for example a tilt sensor or an angle sensor.

The second processor 2 is now described in detail. The second processor 2 is configured to identify user exercise diabetes risk based on the great quantity of data sets collected by the sensor module according to the relationship between the exercise monitoring data it extracts and autonomous behavior. It analyzes the great quantity of collected data sets provided by the sensor module and builds a diabetes prediction model for identifying user exercise diabetes risk.

Therein, the phrase “exercise monitoring data” refers to the real-time data about the user who are performing the exercise regimen. The phrase “autonomous behavior” refers to the capability of the user in the current stage to independently act as output by the second processor 2 according to its evaluation on the great quantity of data related to the user. The autonomous behavior is expressed by at least one exercise capacity evaluation data. The exercise capacity evaluation data describes the capacity of the user in the current stage to independently act. The exercise capacity evaluation data is generated from at least one data user historical exercise monitoring data, such as movement duration, movement range and movement frequency. The autonomous behavior is used as a baseline for identify user exercise diabetes risk. The exercise monitoring data is used to describe the real-time data of exercise capacity of the user who is performing an exercise regimen. According to the relationship between the exercise monitoring data and the autonomous behavior, a load data is calculated as the magnitude by which the exercise capacity real-time data exceeds the historical exercise capacity data. Then the calculated load data is compared with a preset load data threshold, so as to enable prediction of exercise diabetes risk based on the load data.

This is to evaluate how the user in the current stage can independently act before the user takes an exercise regimen, and provide a baseline for identify user exercise diabetes risk. When the real-time user exercise capacity data is generated, it is compared with the evaluation data, so as to determining whether the exercise regimen in effect currently exceeds the preset control conditions. Where it is determined that the exercise regimen exceeds the preset control conditions, dynamic adjustment can be made to the exercise capacity data of the exercise regimen, such as grip requirements, strength requirements, joint movement degree requirements, correspondingly.

The “diabetes prediction model” is used to describe the relationship between the exercise monitoring data and the autonomous behavior. The model is preset with a load data threshold and it adjusts the load data threshold according to user operation. Alternatively, the load data threshold is automatically adjusted according to the big data analysis results provided by the second processor 2.

However, in the process of building the diabetes prediction model, since the search space defined by the great quantity of collected data sets provided by the sensor module is quite large, the computing speed of the second processor 2 for evaluation can be significantly reduced. In addition, the collected data sets coming from the sensor module always contain some data irrelevant to user exercise capacity and/or diabetes risk, and it takes additional time to remove these irrelevant terms, thus adding complexity and feedback duration to the evaluation performed by the second processor 2.

In order to address the aforementioned shortcomings of the prior art, preferably, the relationship between the exercise monitoring data to be extracted and the autonomous behavior is determined according to salient features screened out using machine learning. Before analysis of the relationship between the exercise monitoring data and the autonomous behavior, irrelevant features are skimmed off from all the features, so as to identify the salient features that are highly relevant to diabetes risk.

The second processor 2 is now described in detail. The second processor 2 first acquires a great quantity of diabetes diagnosis case samples by means of performing information interaction with other smart electronic devices 4, and then screens out several salient features of the exercise regimen that are more relevant to diabetes risk according to machine learning. Therein, the salient features screened out according to machine learning refer to an output set generated by imputing training sets to a feature-weight-based LARS diabetes model as one described in Embodiment 1.

The training set refers to a great quantity of diabetes diagnosis case samples. Each case sample at least comprises its exercise regimen data, such as sedentariness duration, exercise intensity, exercise time, exercise duration, exercise frequency, exercise type, diabetes risk changing tendency. For example, for the exercise regimens of a certain case sample performed in a certain period of time, the diabetes risk changing tendency is determined according to variations of diabetes risk criteria before and after the exercise regimens are performed. These diabetes risk criteria may include a blood sugar peak, a heart rate peak, a blood pressure peak and so on. Then a Lasso regression model is defined by inputting some exercise regimen data as candidate features (also referred to as a diabetes data set feature matrix), and the diabetes risk changing tendency is defined as a screening target (also referred to as an actual result label). Afterward, the feature-weight-based LARS algorithm is used to solve the model, thereby outputting the screened salient features and the regression coefficient values corresponding thereto.

With the feature-weight-based LARS algorithm that is superior to the standard LARS in both approaching speed and accuracy, the screened salient features can effectively skim off terms irrelevant to exercise capacity and/or diabetes risk from the data, thereby reducing complexity and feedback duration of real-time evaluation performed by the diabetes prediction model.

Based on the determined salient features, the second processor 2 uses the data sets collected by the sensor module to identify user exercise diabetes risk according to the relationship between the exercise monitoring data extracted through machine learning and the autonomous behavior.

Preferably, the exercise-regimen-adjusting module is configured to dynamically adjust the configuration parameters in the exercise regimens according to the relationship between the exercise monitoring data and the autonomous behavior.

### Embodiment 3

Referring to FIG. 8, the Pima diabetes data set is used herein. In view that the existing k-means algorithm uses initial clustering centroids that are randomly selected and tends to produce inconsistent clustering results, selection of initial clustering centroids has to be such improved that the selected centroids fall in central portion of individual clusters. Therein, the Pima diabetes data set refers to the Pima Indian Diabetes data set in the machine learning database maintained by University of California, Irvine (UCI) as extensively applied by the public.

The system of the present invention serves to build a diabetes prediction model. The system at least comprises a memory and a first processor 1. The first processor 1 is coupled to the memory and is configured to execute at least one step of a diabetes early warning method based on improved k-means clustering. Therein, the diabetes early warning method based on improved k-means clustering at least comprises at least one of the following steps:

(1) Selection of a first clustering centroid: for a certain data set, defining a clustering cluster number k and a radius ε, and selecting a point at which the sum of the distances between the sample point x, and the sample is the greatest as the first clustering centroid;

(2) Selection of a new clustering centroid: calculating the sum Sum(D(x)) of distances between individual sample points and their closest clustering centroids, taking a random value Random in Sum(D(x)), calculating Random−=D(x) until Random≤0, and making selection to obtain the new clustering centroid;

(3) Traversal operation: repeating the previous step until the required k centroids are obtained, written as {μⱼ,j=1, . . . ,k};

(4) Cluster label: calculating the distance between every sample and the clustering centroid, determining a cluster label for the sample according to the minimum distance, and placing the sample into a relevant cluster;

(5) Updating: updating all the clustering centroids; and

(6) Diabetes early warning model: obtaining stable centroids for individual clusters, and putting them in a diabetes piecewise function, thereby obtaining a diabetes early warning model.

The use of the improved k-means clustering algorithm effectively addresses the problem about inconsistent clustering results. By combining the improved k-means clustering algorithm and the diabetes piecewise function, the method for improving the k-means clustering diabetes early warning model has enhanced capability for diabetes early warning and provides a basis for diagnosis and treatment for diabetes at different stages.

The foregoing steps are now further described.

The first step includes defining a clustering cluster number k and a radius ε, calculating a distance dist(x) between each said point and the first clustering centroid, and selecting the point having the greater dist(x) as a new clustering centroid. This is about summing up the individual dist(x) to get sumᵢ=sumᵢ+distᵢ, where i is the number of clustering centroids.

The maximum Sum(dist(x)) is the first clustering centroid, i.e. sum_max=max(sumi).

To select a new clustering centroid, the distance between every point and the first clustering centroid is calculated as dist(x), and the point having the greater dist(x) is taken as a new clustering centroid. The individual dist(x)s are summed up to obtain Sum(dist(x)), and then a random value Random is picked from Sum(dist(x)). Repeative calculation is performed using the equation: Random=Random−dist(x).

When Random≤0, the point is the next clustering centroid, ensuring that dist(x) with the larger distance is more probable to be selected, and the required k centroids are written as {μj,j=1, . . . ,k}.

For labeling a sample cluster, the distance dist_{od} between every sample xᵢ and the clustering centroid {μj,j=1, . . . ,k} is calculated. Then a cluster label λᵢ is determined for the sample Xᵢ according to the minimum distance, before the sample Xᵢ is placed into a relevant said cluster:

C_{λ}_{ᵢ}=C_{λ}_{ᵢ}∪{Xᵢ}.

All the clustering centroids are updated by calculating all the new clustering centroids using the following equation:

$${μ}_{i}^{′} = \frac{1}{\vert {C}_{i} \vert} {\sum }_{x ∈ {C}_{i}} x .$$

The step of building the diabetes early warning model involves obtaining stable centroids for individual clusters according to the foregoing steps, and putting them in a diabetes piecewise function, so as to obtain a diabetes early warning model, wherein the diabetes early warning piecewise function is:

$$y = \{ \begin{matrix}0 & {dist}_{od} ( x - {μ}_{1} ) < {dist}_{od} ( x - {μ}_{2} ) \& {dist}_{od} ( x - {μ}_{1} ) < {dist}_{od} ( x - {μ}_{3} ) \\ 1 & {dist}_{od} ( x - {μ}_{2} ) < {dist}_{od} ( x - {μ}_{1} ) \& {dist}_{od} ( x - {μ}_{2} ) < {dist}_{od} ( x - {μ}_{3} ) \\ 2 & {dist}_{od} ( x - {μ}_{3} ) < {dist}_{od} ( x - {μ}_{2} ) \& {dist}_{od} ( x - {μ}_{3} ) < {dist}_{od} ( x - {μ}_{1} )\end{matrix} .$$

where, μᵢ(i=1,2,3) is the i^{th} clustering centroid, while y=0, y=1, and y=2 represent Healthy, Stage I Warning and Stage II Warning, respectively. The early warning model is useful to predict whether a subject is with diabetes and in which stage the patient is.

In order to further verify the effectiveness of the model of the present invention, comparison among the method for improved k-means clustering diabetes early warning model of the present invention, the standard k-means clustering method and the methods described in the non-patent Literature [1] and Literature [2] as recited previously in the document, and evaluation of these methods according to criteria like homogeneity, integrity, FMI, ARI means, CHI, average rate of convergence, average iterations of convergence and algorithm time were made and described below.

Therein, ARI (Adjusted Rand Index) as one of the criteria for evaluating clustering effects is set in the range of [−1,1]. In a broad sense, ARI measures goodness of fit between data distributions, with the larger value indicating the better clustering result. Therein, Literature [1] refers to: PCA-TDKM Algorithm for K-means Initial Clustering Center Optimization by LIU Rong-kai and SUN Zhong-lin [J]. Software Guide, 2018,17(09):85-87. It provides a PCATDKM algorithm that has the conventional K-means algorithm incorporate PCA, TD and maximum & minimum distance algorithms. The PCA algorithm performs dimension reduction on data object sets, thereby speeding up the clustering process. The TD algorithm dynamically select initial clustering centroids according to the actual distribution of data objects, so that the initial k clustering centroids obtained using the clustering algorithm correspond to the actual clustering results. Literature [2] refers to: Yuan Q L, Shi H B, Zhou X F. An optimized initialization center K-means clustering algorithm based on density [C]//IEEE International Conference on Cyber Technology in Automation, Control, and Intelligent Systems (CYBER), Shenyang, IEEE,2015:790-794. It provides a method for optimizing K-means initial centroids. The algorithm uses a density-sensitive similarity measurement to calculate the density of an article. Candidate points are selected by calculating the minimal distance between the point and other points with relatively high density. Afterward, outliers are screened out by combining average density. At last, the initial centroids for the K-means algorithm are determined. According to experimental results, the initial centroids obtained using the algorithm were highly precise and useful to filter out abnormality.

FIG. 8 illustrates how to modify the standard k-means clustering algorithm. To this end, the Pima diabetes data set was used. The data of 240 cases were taken as experiment samples, including 200 cases of training sets and 40 cases of test sets. The algorithms were programmed using python, thereby allowing comparative analysis of the algorithms.

Table 3 shows ARI means of the 5 algorithms (i.e. Standard k-means, Improved k-means, Literature [1], Literature [2], and Agglomerative) operating 300 iterations on the diabetes data set. As shown in Table 1, the ARI means of the models built using the improved k-means algorithm, the Literature [1] algorithm and the Literature [2] algorithm are significantly higher than those with the standard k-means algorithm. Therein, the improved k-means of the present invention and the Literature [2] algorithm both incorporated the concept of density, and the ARI values of the resulting models were better than those with the Literature [1] algorithm. However, both the models built using the standard k-means algorithm and the improved k-means algorithm performed less as well as that built using the Agglomerative algorithm based on density. This is because the density-based Agglomerative algorithm initial clustering centroids are selected after the density-reachable distance parameters are determined, thus contributing to consistent clustering results. Nevertheless, due to its nature, when processing high-dimensional data, the density-based Agglomerative algorithm is inferior to the k-means algorithm in terms of scalability.

TABLE 3
ARI means of different algorithms on the new diabetes data set

| Algorithm | New Diabetes Data Set |
|---|---|
| Improved K-Means | 0.85 |
| Standard K-Means | 0.69 |
| Literature [1] Algorithm | 0.81 |
| Literature [2] Algorithm | 0.85 |
| Agglomerative | 0.92 |

Table 4 displays means for 5 criteria (homogeneity, integrity, FMI, ARI means, and CHI) on the new diabetes data set obtained using the 5 algorithms (i.e. Standard k-means, Improved k-means, Literature [1], Literature [2], and Agglomerative). As shown in Table 2, the model of the present invention performed better than that of the standard k-means algorithm in all the five criteria, and was also superior to the models according to the algorithm of Literature [1] and the Agglomerative algorithm, while slightly better than the model built using the algorithm of Literature [2]. It is clear that as to ARI and CHI, the model of the standard k-means algorithm performed much less as well as the models of all the other four algorithms. However, as to homogeneity, integrity and FMI, the models of all the 5 algorithms performed comparably. This is because the three criteria are mainly used to measure how accurate a clustering result is. While the model built using the standard k-means algorithm had acceptable accuracy on the training set, its instability led to poor distribution in the resulting model, meaning that the model had poor generalization.

TABLE 4
Means of models from different algorithms
on new diabetes data set for 5 criteria

| Algorithm | Homogeneity | Integrity | FMI | ARI | CHI |
|---|---|---|---|---|---|
| Improved K-Means | 0.82 | 0.84 | 0.88 | 0.86 | 961.83 |
| Standard K-Means | 0.77 | 0.81 | 0.81 | 0.71 | 645.41 |
| Literature [1] | 0.80 | 0.82 | 0.85 | 0.83 | 958.74 |
| Algorithm | | | | | |
| Literature [2] | 0.81 | 0.84 | 0.87 | 0.86 | 961.69 |
| Algorithm | | | | | |
| Agglomerative | 0.80 | 0.80 | 0.85 | 0.83 | 957.59 |

In FIG. 9, the axis of ordinate means ARI of one round of clustering, and the axis of abscissa means the number of iterations the algorithm performs in one round of clustering. The mean iteration number and ARI mean for one round of clustering were calculated after three hundred rounds. As shown, the algorithm of the present invention, the Literature [1] algorithm, and the Literature [2] algorithm had higher ARI values when starting to run iterations. With the method for improving selection of initial centroids, the obtained initial clustering centroids were more accurate. The algorithm of the present invention, the Literature [1] algorithm and the Literature [2] algorithm one round of clustering required much fewer iterations in one round of clustering. Therein, the algorithm of the present invention required the least iterations.

Table 5 displays average iterations of convergence and algorithm times related to the models built using 4 algorithms (i.e. the standard k-means, the improved k-means, the Literature [1], and the Literature [2]), respectively. As shown, the number of iterations required by the standard k-means algorithm is basically twice of the other improved algorithms. However, the results of the average algorithm time of one round of clustering indicate that the standard k-means algorithm was not the one taking the longest to solve the model, and both the algorithms of Literatures [1] and [2] used more time than it. This is because the Literature [1] and [2] algorithms added too much mathematical calculation. By doing this, the required number of iteration did reduced, but the algorithm time for every round of clustering increased. While the algorithm of the present invention also had density calculation added, the related operation was performed only one time, more advantageous is that it is not necessary to calculate the entire data set matrix repeatedly with the concept of probability incorporated.

TABLE 5
Average iterations of convergence and algorithm
time of different algorithms on new diabetes data set

| | | Algorithm |
|---|---|---|
| | Iterations of | Time |
| Algorithm | Convergence | (Sec*10⁻³) |
| Improved K-Means | 8.8 | 1.41 |
| Conventional K-Means | 19.4 | 1.47 |
| Literature [1] Algorithm | 11.8 | 1.61 |
| Literature [2] Algorithm | 9.6 | 1.52 |

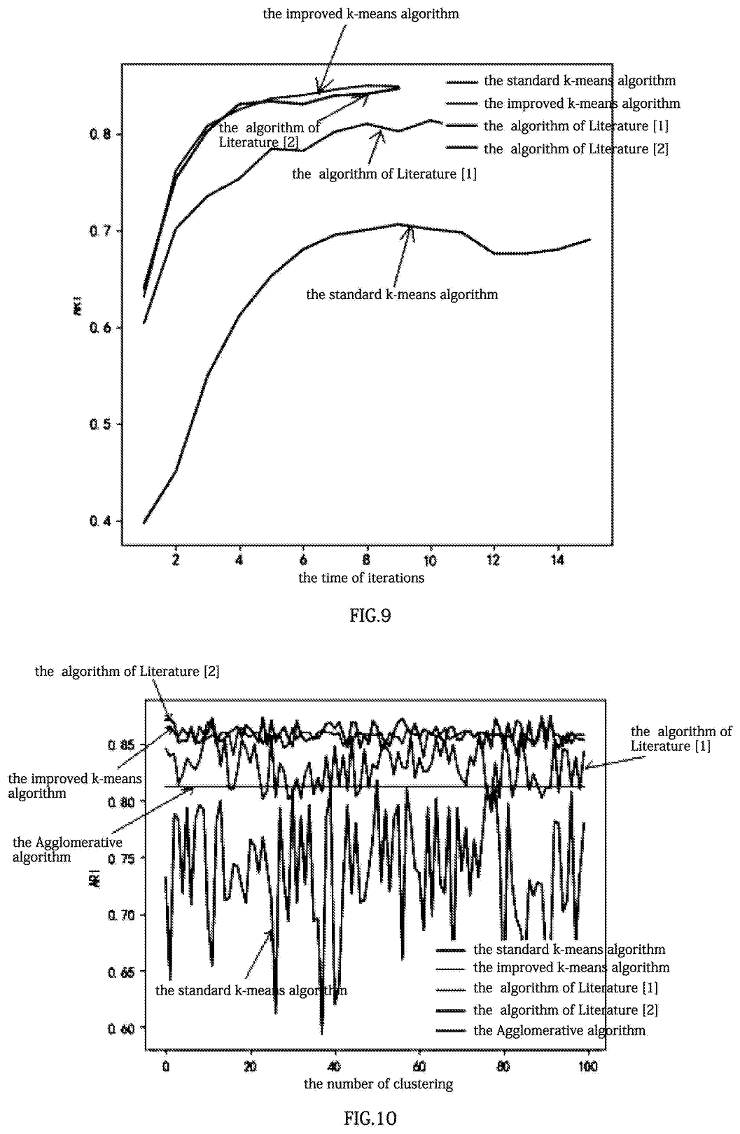

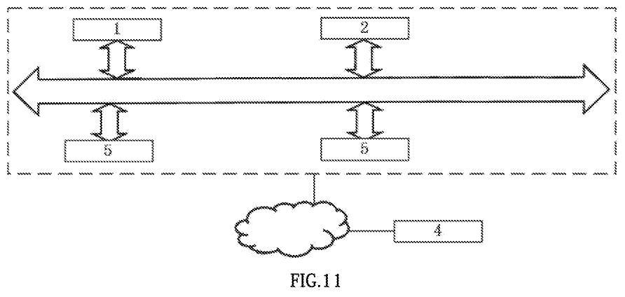

Referring to FIG. 10, the ARI value shown on the axis of ordinate represents the mean of ARI results of the 5 data sets, and the axis of abscissa represents the number of iterations of clustering. As shown in FIG. 10, due to its nature, the Agglomerative algorithm always gave identical results throughout different rounds of clustering, so the resulting pattern in the graph is a line. The results of the model of the standard k-means algorithm display violent fluctuation. The model of the algorithm of the present invention and the model of the Literature [2] algorithm both performed well. As to the variance of the curves, it is 3.19*10⁻⁵ with the algorithm of the present invention, 6.68*10⁻⁵ with the Literature [2] algorithm, 2.94*10⁻⁴, with the Literature [1] algorithm, and 2.78*10⁻³ with the standard k-means algorithm. It is thus clear that the model built using the algorithm of the present invention was the most stable, followed by the model built using the algorithm of Literature [2], and the model built using the standard k-means algorithm was the least stable.

To sum up, the models of all the algorithm of the present invention, the Literature [1] algorithm, the Literature [2] algorithm and the Agglomerative algorithm performed better than the model of the standard k-means algorithm in terms of all the criteria. The algorithm of the present invention provided the best results for the criteria of convergence and algorithm time. The algorithms of Literature [1] and Literature [2] had better convergence as compared to the standard k-means algorithm, but they required longer algorithm time. The models of the algorithm of the present invention, the Literature [1] algorithm, and the Literature [2] algorithm were more stable than the model of the standard k-means algorithm. Therein, the model of the algorithm of the present invention displayed the best stability.

Based on this, the present invention combines the improved k-means clustering algorithm and the diabetes piecewise function to provide a method for improving a k-means clustering diabetes early warning model, thereby addressing the problem about inconsistent k-means algorithm clustering results and improving an early warning model in terms of accuracy and consistency.

### Embodiment 4

The present embodiment provides further improvements to the first processor 1 and second processor 2 of Embodiment 3, and what is identical to its counterpart in the previous embodiment will not be repeated in the following description. Specifically, referring to FIG. 4, the present embodiment provides a diabetes early warning system, the system at least comprises a first processor 1 and a second processor 2 as described in Embodiment 3, a memory coupled to the first processor 1 and the second processor 2, and an interface 5 therebetween.

A user, such as a patient with pre-diabetes, may voluntarily want to take exercise when receiving advice from his/her physician. However, in practice, excessive exercise and bad exercise timing are common lapses that can bring about risk of injury. The existing wearable smart devices and management systems for patients with diabetes as those disclosed in some patent documents are all designed to monitor physiological information of their users and to give alerts when abnormality in physiological information is detected. Variations in physiological information are mainly caused by user actions. These known device and systems tend to have problems about serious monitoring lag and high data sensitivity, making them unable to provide their users with timely and reliable diabetes risk control. Different from the foregoing prior-art articles, the diabetes early warning system of the present invention proactively determines whether a user faces potential risk due to his/her actions before abnormality appears in his/her physiological information. Based on user actions that are highly related to individual differences, diabetes risk is hierarchically controlled at the level of exercise intervention, so as to enhance therapeutic effects and eliminate problems about serious monitoring lag and high data sensitivity.

The diabetes early warning system can be used by individual users to control diabetes risk, especially to control diabetes risk raising during physical exercise. Therein, the term “user” may include a patient with pre-diabetes and/or a diabetic. A patient with pre-diabetes refers to an individual in which early-stage diabetes tends to develop. The phrase “diabetes risk” includes risk of developing from pre-diabetes to diabetes and/or risk of pathophysiology of diabetes. The diabetes early warning system may be implemented in the form of a wearable smart device, a smart mobile terminal or the like.

The diabetes early warning system includes a first processor 1. The first processor 1 is configured to use a diabetes early warning model to predict whether a user is with diabetes and in which stage his/her diabetes is. The prediction result is one of Healthy, Stage I Warning, and Stage II Warning.

The diabetes early warning system further comprises an exercise-regimen-generating module. The exercise-regimen-generating module serves to acquire exercise monitoring data related to the user and to execute Stage I risk warning according to the user data, so as to determine an exercise risk model related to the user. The “exercise monitoring data” at least comprises sedentariness duration, exercise intensity, exercise time, exercise duration, exercise frequency, exercise type and so on. The exercise monitoring data is acquired through information interaction between the exercise-regimen-generating module and other smart electronic devices 4. The “user data” at least comprises the diabetes stage the user is in, diet monitoring data, medicine monitoring data, medical history data, geographic location information, physiological monitoring data, and physical fitness evaluation data and so on. The user data may be acquired through information interaction between the exercise-regimen-generating module and other smart electronic devices 4. The “diet monitoring data” may be acquired through analyzing pictures of diet taken by the user, or may be determined using information of diet time, food type and food quantity input to the smart mobile terminal by the user. Similarly, the “medicine monitoring data” may be determined using information of medication therapy programs of the user and time of taking medicine recorded by the user. The “medical history data” includes complications the user has, exercise therapy programs recommended by physicians, medication therapy programs and so on. The foregoing exercise-regimen-generating module/smart electronic device 4 may be a wearable smart device, such as a smart bracelet, or a smart mobile terminal, such as a smartphone. The “physical fitness evaluation data” may be a body mass index also known as BMI. BMI (in kg/m²) is determined by dividing the body weight (in kg) by the square of the body height (in m).

The following description with reference to “Stage I risk warning” will further explain the solution provided by the present invention to problems about serious monitoring lag and high data sensitivity.

Stage I risk warning is triggered when the exercise-regimen-generating module performs analysis and accordingly confirms that the exercise monitoring data of the user exceeds the preset risk range and the physiological monitoring data does not exceed the preset risk range. In other words, when the physiological monitoring data does not exceed the preset risk range, or in other words, when it is impossible to determine whether there is potential risk for the user in the current state according to the physiological information, the exercise-regimen-generating module continues to monitor the exercise state of the user, and analyzes the acquired exercise monitoring data and physiological monitoring data. From the perspective of prevention, monitoring exercise actions has priority over monitoring physiological information abnormality.

When the exercise monitoring data exceeds the preset risk range, which means that the current exercise actions of the user may cause potential risk, Stage I risk warning is executed to determine the exercise risk model adaptive to the user. Therein, the preset risk range includes preset threshold ranges corresponding to meal time, sedentariness duration, exercise amount of the day, and exercise amplitude, respectively. The preset risk range may be dynamically changing values set according to individual differences of different users, respectively. For example, blood sugar abnormality usually appears 1-2 hours after a meal. As another example, even high-intensity physical exercise cannot offset the negative effects of sedentariness. As a further example, different exercise types each have appropriate exercise duration or the statistical exercise amount finished by the user in the current day or preset body movement amplitude and duration. Based on these, timely monitoring on body movement with excessive amplitude conducted by the user can be achieved. The preset risk range is a restrictive condition acting as a limit for the user to keep away so as to prevent further diabetes risk. A situation going beyond the preset risk range does not trigger warning. In this way, diabetes risk monitoring that satisfies requirements for timeliness and data sensitivity at the same time can be achieved.

As long as a diabetic takes exercise, whether it be aerobic or anaerobic, hypoglycemic effects appear and low blood sugar risk may also come. Particularly, after a diabetic takes hypoglycemic agent or receives insulin injection, when the hypoglycemic effects caused by medicine and caused by exercise synergize with each other, it is most likely to cause low blood sugar. To be specific, if a diabetic takes exercise in less than half an hour after he/she receives insulin injection or takes hypoglycemic medicine, his/her body can absorb hypoglycemic medicine at an increased velocity, making low blood sugar more likely to happen. Besides, in addition to high blood sugar, a diabetic usually has hypertension and dyslipidemia as complications. If diabetes and its comorbidities are not well controlled for a long term, other complications may ensue, such as kidney diseases, neurological disorders, cardiovascular diseases, retinopathy, musculoskeletal disorders and so on. In the event that the user has complications of different diseases, it is necessary to tailor a personalized exercise risk model/exercise monitoring scheme for the user. For example, for a patient with minor retinopathy as a complication, he-she can choose aerobic exercise of low-to-middle intensity, and avoid activities that require the user to hold his/her breath, such as weightlifting. A patient with moderate retinopathy can also choose exercise of low-to-middle intensity, and avoid activities that cause his/her head to exert force downward. A patient with major retinopathy is subject to fundus hemorrhage, and thus must be careful about exercise. It is recommended that such a patient only take some certain low-intensity exercise.

With Stage I risk warning, the early warning system of the present invention preferentially analyzes current exercise data of a user against Stage I risk warning conditions determined according to certain user data, especially medical history data. The conditions for Stage I risk warning is determined according to medical history data in the user data that is associated with exercise data.

The medical history data includes complication disease history (such as complication type, complication severity, complication frequency and so on), medication therapy programs (such as time of taking hypoglycemic medicine, dosage of hypoglycemic medicine, insulin injection time, insulin injection dosage, and so on), and preliminary diabetes prediction results determined by the first processor 1. The diabetes early warning system has some Stage I risk warning conditions preloaded in its memory. The conditions include at least one attribute. According to the attributes of the Stage I risk warning conditions, at least one exercise risk model stored in advance can be called. Several attributes correspond to at least one feature.

The features are different types of the user data, and may be one or several of in which diabetes stage the user is, diet monitoring data, medicine monitoring data, medical history data, geographic location information, physiological monitoring data, and physical fitness evaluation data. An attribute refers to the restrictive condition of at least one exercise monitoring data corresponding to each feature. Herein, the attributes include exercise capacity level and exercise regimen level.

The relationship between the foregoing features and attributes is now explained with reference to some examples. In one example, the feature of diabetes preliminary prediction results determined by the first processor 1 corresponds to the attribute of the exercise regimen level. If the diabetes preliminary prediction result determined by the first processor 1 is Stage II Warning, the attribute of its exercise regimen level is Level A (or in a numeral form). For example, a feature of the complication type corresponds to an attribute of an exercise regimen level. For a feature such as a moderate retinopathy complication, the attribute of its exercise regimen level is Level B (or in a numeral form). If a restrictive condition of an attribute corresponding to a certain feature is higher than the restrictive condition of the same attribute corresponding to other features, the higher restrictive condition prevails, so as to give comprehensive consideration to potential diabetes risk or aggravation risk of the user. For example, for a feature of moderate retinopathy as a complication, the attribute of its exercise regimen level is Level B (or in a numeral form). As to the feature of the medication therapy program, when the time the user takes hypoglycemic medicine does not exceed the preset hypoglycemic medicine taking duration, which means that the hypoglycemic effect of the medicine still lasts, the attribute of its exercise regimen level is Level A (or in a numeral form), and Level A is taken as the exercise regimen level of the user. For the feature of physical fitness evaluation data, when the physical fitness evaluation data of the user indicates a state of being substandard, its exercise capacity level is Level C.

The coincidence relationship between Stage I risk monitoring condition and the exercise risk model is now described with reference to a particular example. As described previously, when it is determined that the user corresponds to the Stage I risk monitoring condition that includes the exercise capacity level, Level C, and the exercise regimen level, Level A, as two attributes, it is determined that an exercise risk model with normal exercise intensity is suitable for the user at present. The exercise risk model of normal exercise intensity includes plural exercise regimens, such as standing, walking, and housekeeping. Each exercise regimen includes sedentariness duration, exercise intensity, exercise time, exercise duration, exercise frequency and the proper control ranges corresponding to the above, respectively.

At this time, the exercise-regimen-generating module obtains several exercise regimens that fit the user. One more issue to address is individual differences among users. Different users adapt to different exercise regimens differently due to their different physical conditions. Therefore, the present invention further analyzes the current exercise regimen using Stage II risk warning, with adequate consideration to individual differences among users, so as to provide the user with an exercise therapy program that is safer and more effective by hierarchically controlling diabetes risk at the level of exercise intervention.

The exercise-regimen-generating module is further configured to execute Stage II risk warning analysis when the current exercise data of the user does not satisfy the Stage I risk warning conditions. Specifically, the exercise-regimen-generating module is further configured to determine the relationship between the current exercise monitoring data and the physiological information of the user according to the statistical changing tendency curve between the historical physiological information and the historical exercise monitoring data of the user, to acquire the current physiological information related to the user provide by the smart electronic device 4, and to perform trend analysis between the current physiological information and the determined relationship, so as to determine an estimate of the physiological information that will be true if the user continues to conduct the current exercise, to determine at least one exercise risk warning and/or exercise guidance scheme that is generated by the exercise risk model after the exercise regimens determined to be restricted according to the prediction physiological information of the user have been removed according to the prediction physiological information obtained from the trend analysis, and to send an exercise risk warning and/or exercise guidance to the user through a prompt module or other smart electronic devices 4.

Preferably, the exercise risk model refers to a statistical changing tendency curve between the historical physiological information and the historical exercise monitoring data that is determined according to the user data. The statistical changing tendency curve intuitively reflects the changing tendency of the physiological information of the user when he/she conducts different types of exercise, and serves as a basis for analysis and prediction of subsequent exercise of the user. The exercise-regimen-generating module generates the exercise intervention information related to the user based on the exercise risk model. The exercise intervention information includes an exercise risk warning and/or exercise guidance. Therein, the exercise intervention information provides intervention and prevention recommendation about the exercise actions of the user in two forms. The first is the exercise risk warning, which informs the user of which exercise or movement may bring about physical risk. This helps the user to prevent this risky movement during his/her further exercise and in his/her daily life, thereby providing short-term and long-term benefits to the treatment for the user. The exercise risk warning and/or exercise guidance is generated on the basis that the exercise regimens determined to be restricted for the user, or the restricted exercise regimens, have been removed.

The restricted exercise regimen is determined according to the prediction physiological information of the user. During the analysis for determining the restricted exercise regimens is conducted, the two attributes, namely the exercise therapy program/exercise capacity level and the exercise regimen level that disfavor to the user are acquired. Accordingly, the components in of the acquired exercise risk model related to the user that satisfy the restricted exercise regimens are completely removed. Then the exercise risk model is updated with the removed and remaining exercise regimen, and fed back to the user for his/her reference. The prediction physiological information is obtained by performing trend analysis between the current physiological information related to the user and the relationship. Therein, the prediction physiological information refers to the estimate of the physiological information estimated with the assumption that the user continues to conduct the current exercise. The relationship is determined according to the statistical changing tendency curve between the historical physiological information of the user and the historical exercise monitoring data. The relationship refers to prediction of the changing tendency between the current exercise monitoring data and the physiological information of the user.

As a preferred mode, the early warning system of the present embodiment further comprises a sensor module such as the one described in Embodiment 2, a second processor 2 and an exercise-regimen-adjusting module. The sensor module is configured to collect the initial data of the diabetes early warning system, the parameters used in the system, and the user data related to the user. The second processor 2 is configured to: when it is detected that the user is executing the exercise therapy program generated by the foregoing exercise-regimen-generating module and that the prediction result related to the user generated by the first processor 1 is Stage II, use the data set collected by the sensor module to identify user exercise diabetes risk according to the salient features screened out through machine learning and the relationship between the extracted exercise monitoring data and the autonomous behavior. The exercise-regimen-adjusting module is configured to dynamically adjust the configuration parameters of the exercise regimens by identifying the exercise diabetes risk according to the relationship between the exercise monitoring data and the autonomous behavior determined through the analysis conducted by the second processor 2.

As a preferred mode, the system of the present invention includes: a smart electronic device 4 operated or worn by a user. The smart electronic device 4 is equipped with a first processor 1, a second processor 2, an exercise-regimen-generating module, a sensor module, an exercise-regimen-adjusting module and so on. Plural processors provided on a smart electronic device operated or worn by the user communicate with a smart electronic device operated or worn by the user/care giver through a computer network. The first processor 1, the second processor 2, the exercise-regimen-generating module, the exercise-regimen-adjusting module and other processors may be interconnected each other through for example a communication bus (a physical line) of a motherboard. Preferably, the first processor 1 and at least one smart electronic device (such as a sensor module) send the data they have processed to the exercise-regimen-generating module and the second processor 2. The exercise-regimen-generating module caches the data and sends the data it has processed to at least one smart electronic device (such as a smartphone operated by the user) or a prompt module, and the exercise-regimen-adjusting module. The exercise-regimen-generating module sends the generated exercise regimen to the exercise-regimen-adjusting module. The exercise-regimen-adjusting module dynamically adjusts its configuration parameters according to the generated exercise regimen so as to optimize the exercise regimen for the user. The exercise-regimen-adjusting module processes data it receives and sends the resulting data to at least one smart electronic device (such as a smartphone operated by the user) or a prompt module, and the exercise-regimen-generating module.

The present invention has been described with reference to the preferred embodiments and it is understood that the embodiments are not intended to limit the scope of the present invention. Moreover, as the contents disclosed herein should be readily understood and can be implemented by a person skilled in the art, all equivalent changes or modifications which do not depart from the concept of the present invention should be encompassed by the appended claims.

What is claimed is:

1. A diabetes risk early warning system, wherein the system comprises:
a memory; and
a first processor (1), which is based on improved k-means clustering, coupled to the memory, and configured to:
according to selected first clustering centroids, obtain stable centroids for individual clusters, and put them into a diabetes piecewise function, thereby obtaining a diabetes early warning model,
wherein the first clustering centroid is selected by selecting a data set, defining a clustering cluster number k and a neighborhood radius ε, and selecting a sample point on which a sum of distances between a sample point Xᵢ and a sample is the greatest as the first clustering centroid, so as to make the first clustering centroid fall in a central portion of the respective clusters.

2. The diabetes risk early warning system of claim 1, wherein the step of selecting the point on which the sum of the distances between the sample point Xᵢ and the samples is achieved through at least one of the following steps:
calculating a distance dist(x) between each said point and the first clustering centroid;
selecting the point having the greater dist(x) as a new clustering centroid;
summing up the individual dist(x); and
identifying a Sum(dist(x)) that is the greatest as the first clustering centroid.

3. The diabetes risk early warning system of claim 2, wherein the first processor (1) is further configured to:
make selection to obtain the new clustering centroid,
wherein a point with a greater distance between the sample point Xᵢ and the first clustering centroid is selected as the new clustering centroid.

4. The diabetes risk early warning system of claim 3, wherein the step of selecting the point with a greater distance between the sample point Xᵢ and the first clustering centroid as the new clustering centroid is achieved through at least one of the following steps:
calculating a distance dist(x) between each said point and the first clustering centroid;
selecting the point having the greater dist(x) as a new clustering centroid;
summing up the individual dist(x) to obtain Sum(dist(x));
picking up a random value Random from Sum(dist(x));
performing repeative calculation using an equation: Random=Random−dist(x); and
taking a point on which Random≤0 as the next clustering centroid.

5. The diabetes risk early warning system of claim 4, wherein the first processor (1) is further configured to:
perform traversal, in which Step 2 is repeatedly performed until the required k centroids are obtained, written as {μⱼ,j=1, . . . ,k}.

6. The diabetes risk early warning system of claim 5, wherein the first processor (1) is further configured to:
tag a sample cluster,
which includes calculating a distance dist_{od} between each said sample Xᵢ and the clustering centroids {μⱼ,j=1, . . . ,k}, determining a cluster label λᵢ for the sample Xᵢ according to the minimum distance, and placing the sample Xᵢ into a relevant said cluster:
C_{λ}_{ᵢ}=C_{λ}_{ᵢ}∪{xᵢ}.

7. The diabetes risk early warning system of claim 6, wherein the first processor (1) is further configured to:
perform updating,
in which all the clustering centroids are updated, and all the new clustering centroids are calculated using the following equation:
$${μ}_{i}^{′} = \frac{1}{\vert {C}_{i} \vert} {Σ}_{x ∈ {C}_{i}} x .$$

8. The diabetes risk early warning system of claim 7, wherein the step of updating all the clustering centroids is achieved through at least one of the following steps:
calculating
$${μ}_{i}^{′} = \frac{1}{\vert {C}_{i} \vert} {Σ}_{x ∈ {C}_{i}} x ,$$
and determining whether uᵢ′=uᵢ is true; and
if yes, remaining the current centroid unchanged; or
if no, updating the current uᵢ with uᵢ′.

9. The diabetes risk early warning system of claim 8, wherein the diabetes early warning piecewise function is:
$$y = \{ \begin{matrix}0 & {dist}_{od} ( x - {μ}_{1} ) < {dist}_{od} ( x - {μ}_{2} ) \& {dist}_{od} ( x - {μ}_{1} ) < {dist}_{od} ( x - {μ}_{3} ) \\ 1 & {dist}_{od} ( x - {μ}_{2} ) < {dist}_{od} ( x - {μ}_{1} ) \& {dist}_{od} ( x - {μ}_{2} ) < {dist}_{od} ( x - {μ}_{3} ) \\ 2 & {dist}_{od} ( x - {μ}_{3} ) < {dist}_{od} ( x - {μ}_{2} ) \& {dist}_{od} ( x - {μ}_{3} ) < {dist}_{od} ( x - {μ}_{1} )\end{matrix} ,$$
where μᵢ(i=1,2,3) is the i^{th} clustering centroid while y=0, y=1, and y=2 represent Healthy, Stage I Warning and Stage II Warning, respectively, so that the early warning model can be used to predict whether a subject is with diabetes and in which stage the patient is.

10. A diabetes risk early warning system, comprising:
a memory;
a second processor (2), which is based on a feature weight, coupled to the memory, and configured to:
calculate an independent variable feature weight vector and an original relationship vector; and
based on the independent variable feature weight vector and the original relationship vector, output a regression coefficient ω of a LARS diabetes model based on the feature weight.

11. The diabetes risk early warning system of claim 10, wherein calculating the feature weight of the feature independent variable is achieved using the following equation:
$${β}_{i} = \frac{{φ}_{i}}{{\sum }_{k = 1}^{n} {φ}_{k}} ,$$
where φᵢ is an eigenvalue of a characteristic equation |φI−R|=0.

12. The diabetes risk early warning system of claim 11, wherein R in the characteristic equation is a covariance matrix of a diabetes data set matrix X, and is calculated using the following equation:
$$R = [ \begin{matrix}{r}_{11} & {r}_{12} & ⋯ & {r}_{1 n} \\ {r}_{21} & {r}_{22} & ⋯ & {r}_{2 n} \\ ⋮ & ⋮ & ⋱ & ⋮ \\ {r}_{m 1} & {r}_{m 2} & ⋯ & {r}_{mn}\end{matrix} ] ,$$
where,
$${r}_{ij} = {r}_{ji} = \frac{{\sum }_{k = 1}^{m} ( {x}_{ki} - {θ}_{i} ) ( {x}_{kj} - {θ}_{j} )}{\sqrt{{\sum }_{k = 1}^{m} {( {x}_{ki} - {θ}_{i} )}^{2} {\sum }_{k = 1}^{m} {( {x}_{kj} - {θ}_{j} )}^{2}}} ,$$
θᵢ is a mean of the i^{th} feature.

13. The diabetes risk early warning system of claim 12, wherein outputting the regression coefficient ω based on the independent variable feature weight vector and the original relationship vector is achieved through at least one of the following steps:
calculating an angle bisector vector, a regression coefficient vector, a new relationship vector and a maximum relationship;
updating the regression coefficient vector, an estimate vector, a residual vector and an index set; and
determining whether an L2 norm of the residual vector is smaller than a tolerance, and ending if yes, or repeating the above steps if no.

14. The diabetes risk early warning system of claim 13, wherein an angle bisector line u_{A} of a row vector X_{A} is obtained using the following equations:
G_{A}=X^{T}_{A}X_{A}, A_{A}=(1^{T}_{A}G⁻¹_{A}1_{A})^{-1/2},
ω_{A}=A_{A}G⁻¹_{A}1_{A}, u_{A}=X_{A}ω_{A}.

15. A diabetes risk early warning system, comprising:
a memory;
at least one processor, which is coupled to the memory and configured to:
according to selected first clustering centroids, obtain stable centroids for individual clusters, and put them into a diabetes piecewise function, so as to obtain a diabetes prediction model, in which the first clustering centroid is selected by selecting a data set, defining a clustering cluster number k and a radius ε, and selecting a point on which a sum of distances between a sample point Xi and samples, so as to make the first clustering centroid fall in a central portion of the corresponding cluster;
calculate an independent variable feature weight vector and an original relationship vector; and
based on the independent variable feature weight vector and the original relationship vector, output a regression coefficient ω of the diabetes prediction model.
